(19) United States
(12) Patent Application Publication
(10) Pub. No.: US 2007/0070292 A1
(43) Pub. Date: Mar. 29, 2007

(54) Methods and apparatus for comprehensive vision diagnosis

(75) Inventor: Junzhong Liang, Fremont, CA (US)

Correspondence Address: JUNZHONG LIANG, 45 KOOTENAI DRIVE, FREMONT, CA, 94539, US

(73) Assignee: Advanced Vision Engineering, inc., Fremont, CA (US)

(21) Appl. No.: 11/522,254

(22) Filed: Sep. 15, 2006

Related U.S. Application Data
(60) Provisional application No. 60/718,858, filed on Sep. 19, 2005.

(51) Int. Cl. A61B 3/10

(52) U.S. Cl. 351/205

(57) ABSTRACT

A wavefront sensing system for measuring wave aberration of an eye comprises an illumination light source configured to produce a compact light source at the retina of the eye, a small opaque stop configured to block corneal reflection of the illumination light, a wavefront sensor configured to measure the outgoing wavefront originated from the compact light source at the retina. Measuring wave aberration of an eye can be improved by using a Hartmann-Shack sensor with a fixed, localized mark on the lenslet array for unique identification of each focus spot of the sensor to its corresponding lenslet, and by including a refractive correction module and a wavefront fusing algorithms for the determination of wave aberration of an at its far accommodation point. In an additional aspect, a wavefront sensing system is designed to provide more comprehensive diagnosis of refractive corrections by measuring light scattering in the eye as well as wavefront data of lenses used for refractive corrections.

### CROSS-REFERENCES TO RELATED INVENTIONS

The present invention claims priority to the provisional U.S. patent application No. 60/718,858, titled “Methods and Apparatus for Comprehensive Diagnosis of Human Vision,” filing on Sep. 19, 2005 by J. Liang. The present invention is related to commonly assigned U.S. patent application Ser. No. 11/293,611, titled “Methods and Apparatus for Wavefront Sensing of Human Eyes” filed on Dec. 2, 2005 by J. Liang, U.S. patent application Ser. No. 11/293,612, titled “Methods and systems for wavefront analysis” filed on Dec. 2, 2005 by J. Liang and D. Zhu, U.S. patent application Ser. No. 11/371,288, titled “Algorithms and Methods for Determining Aberration-Induced Vision Symptoms in the Eye from Wave Aberration,” filed on Mar. 8, 2006 by J. Liang, U.S. patent application Ser. No. 11/370,745, titled “Methods for Specifying Image Quality of Human Eyes from Wavefront Measurements,” filed on Mar. 8, 2006 by J. Liang, U.S. patent application Ser. No. 11/432,273, titled “Wavefront Fusion Algorithms for Refractive Vision Correction and Vision Diagnosis,” filed on May 10, 2006 by J. Liang, and U.S. patent application Ser. No. 11/432,274, titled “Multitask Vision Architecture for Refractive Vision Corrections,” filed on May 10, 2006 by J. Liang. The disclosures of these related applications are incorporated herein by reference.

### TECHNICAL FIELD

This application relates to systems and methods for refractive vision corrections and refractive vision diagnosis.

### BACKGROUND

Wavefront-guide vision correction is becoming a new frontier for vision and ophthalmology because it offers supernormal vision beyond conventional sphero-cylindrical correction, allows imaging of living photoreceptors, and perfects laser vision correction. Wavefront technology will reshape the eye care industry by enabling custom refractive corrections based on aberrations in individual eyes, reliable vision diagnosis and comprehensive specification of refractive vision corrections.

Wavefront technology is based primarily on precise measurements of eye's wave aberration using a device called wavefront sensors (aberrometers). One popular approach for the wavefront measurement is to measure the outgoing wavefront at the corneal plane using a Hartmann-Shack sensor as described in Liang et al. 94', “Objective measurement of wave aberrations of the human eye with the use of a Hartmann-Shack wave-front sensor,” J. Opt. Soc. Am. A, vol. 11, no. 7, p. 1949, July 1994. FIG. 1 shows a schematic diagram for a typical wavefront system using a lenslet array wavefront sensor. A fixation system 101 assists the tested eye in stabilizing its accommodation and in maintaining the view direction. An illumination light source 102 generates a compact light source to reflect off a beamsplitter (BS2) and shines on the eye's retina as the probing light. The probing light is diffusely reflected by the retina, from which a distorted wavefront is formed at the eye's cornea plane. An optical relay system 103, consisting of lenses (L1) and (L2), relays the outgoing wavefront from the eye and reflected off beamsplitter BS1 to the plane of a lenslet array. A Hartmann-Shack wavefront sensor 104, consisting of a lenslet array and an image sensor, produces a wavefront sensor image as an array of focus spots. An image analysis module 105 detects the focus spots and calculates the wavefront slopes, from which the wavefront is reconstructed by a wavefront estimator 106.

The illumination probing light in FIG. 1 will not only be reflected by the retina but also by the cornea and the crystalline lens. Because of a large change in the refraction index at the first surface of the cornea, the cornea reflex is much stronger than the light reflected from the retina and from the crystalline lens. Therefore, removing corneal reflection of the probing light is critical for wavefront sensing for the eye.

Different approaches were disclosed to address the issue of corneal reflection since the introduction of wavefront sensor. Liang et al 94' described a method of placing an aperture at the conjugate plane of the retina. Because the aperture is conjugate to the retina, it can reduce corneal reflection without affecting the wavefront from the retina. However, corneal reflection around the corneal vertex cannot be eliminated completely because it cannot be separated from the retinal reflection. Williams and Yoon in U.S. Pat. No. 6,264,328B1 described a so-called off-axis approach that uses an illumination beam positioned away from cornea vertex and an aperture placed at the conjugate point of the retina reflection to block the cornea reflex. Although the off-axis approach was described inexpensive, its actual implementation relies on expensive opto-mechanical systems for the correction of focus error in the eye. Without a proper correction for the eye's focus error, the aperture will not only block the corneal reflex, but also the retinal reflection. Additionally, requiring the correction of focus error in the eye before a wavefront measurement makes the wavefront measurement time-consuming if the sphero-cylindrical errors in an eye are not known in advance. A desired approach for blocking the corneal reflection should work indifferently for all eyes without a need for correcting any wavefront error in the eye. Liang and Williams described a method of removing corneal reflection using a polarization beamsplitter in “Aberrations and retinal image quality of the human eye,” J. Opt. Soc. Am. A, vol. 14, no. 11, p. 2873,1997. An illumination light through the polarization beamsplitter produces a linear polarized light as the probing beam into the eye. Because the corneal reflection preserves the polarization direction of the probing beam while the retinal reflection is depolarized, corneal reflection can be removed by the same polarized beamsplitter in the detection arm. The polarization approach is effective for a probing light with a relatively large beam size, but not so effective for illuminations with a beam size smaller than 1 mm, as shown in FIG. 2 with the corneal reflex reflection highlighted in a wavefront sensor image. Another disadvantage of the polarization approach is the loss of about 75% of retinal reflection for the wavefront sensing. It is therefore apparent that a need exists in the art to provide a more effective method for removing cornea reflection in wavefront sensing of an eye. More particularly, the preferred method must be inexpensive, and can block corneal reflex for all eye without correcting for refractive error as a pre-condition.

Wavefront measurements using a Hartmann-Shack sensor require two measurements: one reference measurement from a known reference such as a perfect plane wave and one measurement from the tested object. Every focus spot in a wavefront image of a Hartmann-Shacks sensor has to be uniquely registered to its corresponding lenslets for at least two reasons. First, background errors in the wavefront system are recorded in the reference measurement and can be eliminated. Second, registration of wavefront map to the pupil of eye requires position information of the measured wavefront map in a fixed coordinate system. Unique registration of focus spots without a registration mark in the wavefront sensor was disclosed by using a fixed array of lenslets defined by an aperture in front of a lenslet array in Liang et al. 94'. Wavefront measurement using a fixed array of lenslet is however limited because natural pupil sizes for different eyes vary greatly. Because measuring aberrations in a full natural pupil is important for evaluating night vision, it is therefore apparent that a need exists in the art to provide a wavefront sensor in which each focus spots is uniquely registered to its corresponding lenslet. More particularly, the wavefront sensor must have an unrestricted lenslet array for testing eyes of any pupil size.

Wavefront sensors measure aberration of an eye objectively and the measured wavefront may contain an accommodation offset because tested eyes do not necessarily accommodate at its far accommodation point during a wavefront measurement. Wavefront fusion algorithms were disclosed in U.S. patent application Ser. No. 11/432,273, titled “Wavefront Fusion Algorithms for Refractive Vision Correction and Vision Diagnosis,” filed on May 10, 2006 by Liang to address the issue of accommodation offset. The fusion algorithms rely on data from two devices: a wavefront sensor for wave aberration and a phoroptor for a manifest refraction. A clinical setting using two separate systems is not preferred because it is expensive, time-consuming, and requires more office space. It will be apparent that a need exists in the art to provide a single wavefront-based system with which both the conventional manifest refraction and the high-order aberrations of the eye can be measured quickly and accurately in a cost-effective manner. More particularly, it is highly desired to have a single wavefront system to provide measurements of eye's wave aberration at eye's far accommodation point for reliable vision correction and vision diagnosis.

In addition to measuring wave aberration in an eye, wavefront sensors for the eye can be further configured as a single, cost-effective, mutifunctional workstation for comprehensive vision diagnosis that includes measuring light scattering in the eye and measuring lenses as a lensometer.

Further details of prior eye imaging devices may be found in Liang et al. “Objective measurement of wave aberrations of the human eye with the use of a Hartmann-Shack wave-front sensor,” J. Opt. Soc. Am. A, vol. 11, no. 7, p. 1949, 1994; Liang and Williams “Aberrations and retinal image quality of the human eye,” J. Opt. Soc. Am. A, vol. 14, no. 11, p. 2873, 1997, Westheimer et al. “Evaluating diffusion of light in the eye by objective means” Investigative Ophthalmology & Visual Science, vol. 35, p2652, 1994.

### SUMMARY

The present invention is directed to an apparatus for measuring wave aberration of an eye. The apparatus comprises an illumination light source configured to produce a compact light source at the retina of the eye, a small opaque stop configured to block corneal reflection of the illumination light, and a wavefront sensor configured to measure the outgoing wavefront originated from the compact light source at the retina.

In another aspect, the present invention is directed to a method for wavefront sensing of human eye with a Hartmann-Shack sensor. The method comprises the steps of producing a compact light source at retina of the eye, receiving the light reflected from the retina with a Hartmann-Shack sensor, wherein the Hartmann-Shack sensor contains a fixed, localized mark for the unique identification of each focus spot to its corresponding lenslet, determining coordinates of focus spots in the wavefront image, calculating wavefront slopes from the displacements of each focus spots, and deriving wave aberration of the eye from the calculated wavefront slopes.

In an additional aspect, the present invention is directed to an apparatus for determining a wave aberration of an eye at its far accommodation point. The apparatus comprises a wavefront module configure to measure wave aberration of an eye, a refraction correction module configured for determining a manifest refraction of the eye subjectively, and a wavefront fusion algorithm for the determination of a wave aberration of the eye at its far accommodation point by combining the measured wavefront aberration from the wavefront module and the manifest refraction from the refraction module.

In yet anther aspect, the apparatus for measuring wave aberrations of an eye further includes measuring light diffusion in an eye, comprising a wavefront sensor module configured for measuring wave aberration of the eye, a refractive correction module configured for a refractive correction of conventional sphero-cylindrical error, a double-pass module configured for measuring a double-pass point-spread distribution of the eye, and a metrics for qualifying the light diffusion in the eye based on the data from the double-pass module.

In yet an additional aspect, the apparatus for measuring wave aberrations of an eye further includes measuring lenses as a lensometer, comprising a light source configured to produce a compact light source at the retina when an eye is measured for its aberrations, a second light source configured to produce a wavefront through a lens when the lens is measured, an optical relay for transferring the measured wavefronts to a plane with a wavefront sensor, a Hartmann-Shack sensor for measuring either a wavefront from an eye under test or a wavefront through a lens under test.

The details of one or more embodiments are set forth in the accompanying drawings and in the description below. Other features, objects, and advantages of the invention will become apparent from the description and drawings, and from the claims.

### DRAWING DESCRIPTIONS

FIG. 1 is a schematic diagram of a conventional wavefront system for measuring wave aberration of an eye using a Hartmann-Shack sensor.

FIG. 2 shows an image of a Hartmann-Shack wavefront sensor for an eye that contains an unwanted corneal reflection even though a narrow off-axis light beam is used for producing a compact light source at the retina and a polarized beamsplitter is used for reducing the cornea reflex of the illumination light.

FIG. 3a shows the distribution of light reflected from the retina in a wavefront system with a Hartmann-Shack sensor.

FIG. 3b shows the distribution of light reflected from the corneal of the eye in a wavefront system with a Hartmann-Shack sensor, and a small opaque stop placed near the conjugate plane of the cornea for blocking the corneal reflection during wavefront measurements in accordance with the present invention.

FIG. 3c shows a schematic diagram of a Hartmann-Shack sensor with at least one lenslet blocked by a small opaque stop.

FIG. 4a shows a configuration for blocking corneal reflection with a small opaque stop that is vertex-centered in accordance to the present invention.

FIG. 4b shows a configuration for blocking corneal reflection with a small opaque stop that is placed at an optical image of corneal reflection in a plane conjugate to the cornea in accordance to the present invention.

FIG. 5a shows ambiguity in identifying focus spots of a wavefront image to their corresponding lenslets for an unrestricted lenslet array.

FIG. 5b shows a localized mark, indicated as the removal of at least one lenslet in the lenslet array, for unique identification of focus spots to their corresponding lenslets in the lenslet array in accordance to the present invention.

FIG. 6 shows a schematic diagram of an apparatus for determining a wave aberration of an eye at its far accommodation point in accordance to the present invention.

FIG. 7 shows a schematic diagram of an apparatus for measuring both wave aberration and light diffusion in an eye in accordance to the present invention.

FIG. 8a shows a schematic diagram of a wavefront sensor configured for measuring wave aberration of an eye.

FIG. 8b shows a schematic diagram of a wavefront sensor in FIG. 8a configured as a lensometer.

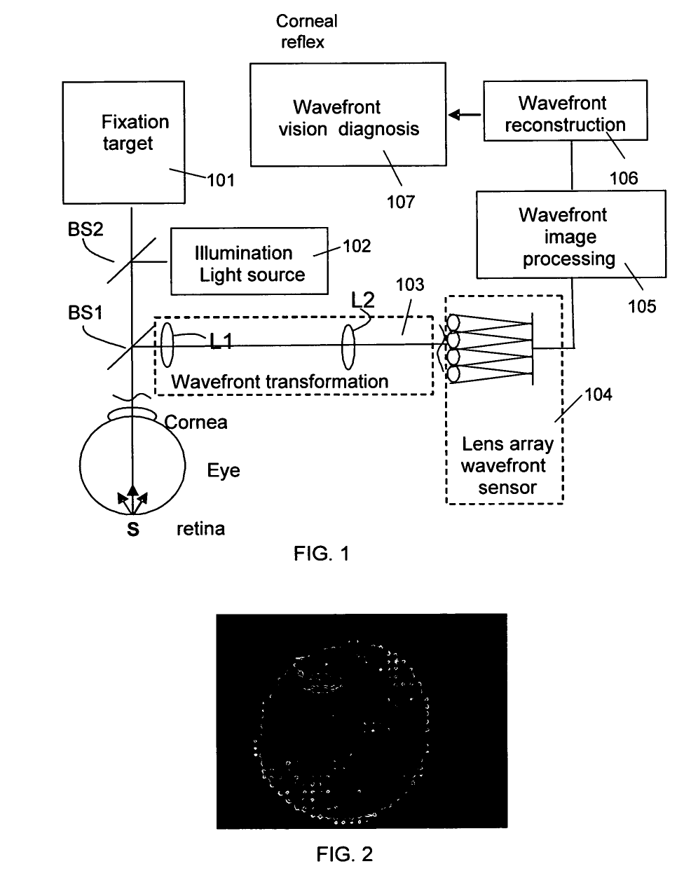

### DETAILED DESCRIPTION

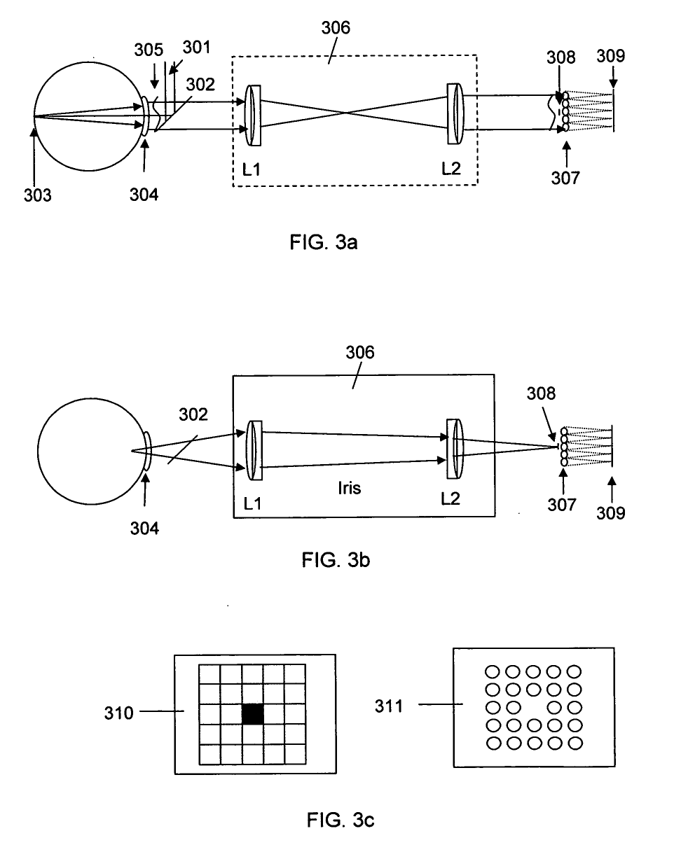

FIG. 3 illustrates an embodiment for blocking corneal reflection with a small opaque stop in a wavefront sensor for an eye. As shown in FIG. 3a, an illumination beam 301 is reflected off a beamsplitter 302 and produces a compact light source at the retina of the eye 303. The retinal reflection 305 fills the entire pupil of the eye because of diffuse reflection by the retina and is a point-like source in planes that is conjugate to the retina. Because the lenslet array 307 is placed at a conjugate plan to the cornea through an optical relay system 306, the beam at the lenslet array plan is a reproduction of the wavefront at the cornea. FIG. 3b shows propagation of the light reflected from the cornea in the same wavefront sensor. The first surface of the cornea 304 functions as a spherical mirror with a curvature radius of about 8 mm. The focal point of the cornea for the reflected light is about 4 mm behind the cornea vertex. When a collimated (or slightly curved) beam is used for the illumination, the cornea reflex is like a virtual point source about 4 mm behind the cornea and forms a point image near the focal point of the lens L2. (In a 4f optical relay system wherein the cornea is at the focal plane of the lens L1 and the lenslet array is at the focal plane of the lens L2, the lenslet array is at a conjugate plane of the cornea.) A small opaque stop 308 placed at the image point of the cornea reflection can effectively block the corneal reflection. If the opaque stop is small enough compared to the pupil size, its impact on the wavefront originated from the retina is negligible. As an illustration, FIG. 3c shows a blocked lenslet in a 2 dimensional lenslet array 310 and a missing focus spot in a wavefront sensor image if the opaque stop blocks only one lenslet in sensing wavefront originated from the retina.

The method for blocking the corneal reflex with a small opaque stop shown in FIG. 3 will function indifferently for eyes with different amount of focus errors. In principle, the beam size at the corneal plane can be very large and the opaque stop can be very small in size. Although the opaque stop is shown next to the lenslet array in FIG. 3a and 3b, it can be placed in a place where corneal reflection is concentrated and near any plane that is optically conjugated to the cornea. By placing the opaque stop at a conjugate location of the corneal reflex, corneal reflex is blocked in the wavefront measurement. Because the image point of the cornea reflex is very close to the lenslet array, the small opaque stop will only block wavefront measured at a limited number of lenslets.

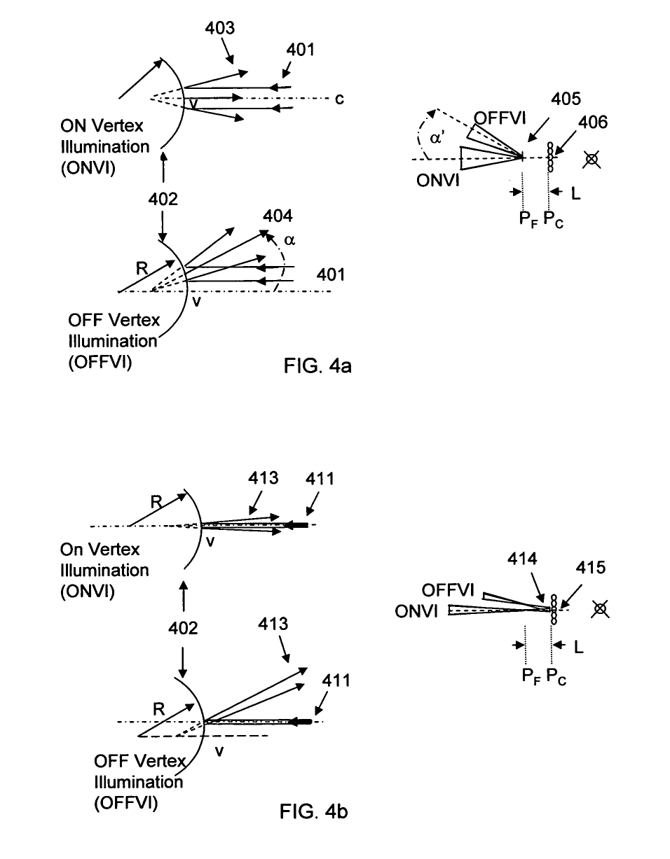

For a more detailed discussion, two cases of probing beams 401 are shown in FIG. 4a: ON-Vertex-Illumination (ONVI) with a beam covering the corneal vertex, and OFF-Vertex Illumination (OFFVI) with a beam away from the corneal vertex. For both cases, the corneal reflection is originated from the same focal point of the corneal sphere but at different angles from the cornea. After being imaged with the optical relay, the image of the corneal reflex for both cases locates at one point near the lenslet array but at different angles of incident. If the illumination beam is parallel to the optical axis of the cornea, the image point will be centered on the axis through the vertex of the cornea. For this reason, we name the method vertex-centered.

For the vertex-centered reflex rejection, a preferred embodiment may include the following features. First, a small opaque stop is placed and bound to an optical flat. The optical flat is chosen because it has no or little impact on the measured wavefront. The opaque stop is small enough (˜0.5 mm) so that at most very few lenslets will be blocked for measuring wavefront from the eye in eye's pupil. Second, the opaque stop can be adjusted with the optical flat in three dimensions in the initial system setup. Along the optical axis, the opaque stop is placed in a conjugate plane of the corneal focal point. In the plane perpendicular to the optical axis, the stop is positioned to block only at most a few fixed lenslets around the optical axis. Third, an alignment mark capable of indicating the location of the opaque stop in the corneal plane is placed in the live images of a pupil camera for pupil alignments. Fourth, the vertex of the cornea is aligned so that the opaque stop can block the corneal reflex. Fifth, wavefront measurements at the missing points can be interpolated or extrapolated according the wavefront slopes next to the missing sampling locations.

Even though collimated illumination beams are illustrated in FIG. 3 and FIG. 4, our proposed method also works for slightly curved illumination beams or beams with slightly off-axis retina illumination. For non-collimated beams, the image of corneal reflection will locates at one point very close to the focal point of the cornea because the cornea has far greater refractive power than that of the illumination beam at the cornea.

FIG. 4b shows another embodiment of blocking the corneal reflex with an opaque stop 414 when a narrow beam 411 is used for producing a compact light source at the retina. Two cases of probing beams are also considered: ON-Vertex-Illumination (ONVI) with a small beam covering the corneal vertex, and OFF-Vertex Illumination (OFFVI) with a small beam not covering the vertex. For both cases, the beam size at the corneal plane is the same for the incoming illumination beam and for the reflected beam, but at different angles of incidence. After imaged through the relay system (L1 and L2), the images of the corneal reflex is fixed and determined by the position of the illumination beam in the wavefront sensor. If a small opaque stop is placed at the conjugate plane of the cornea and covers the image of the corneal reflex entirely, the corneal reflex can be blocked completely. We call this method beam-conjugated reflex rejection because it uses an opaque stop at a conjugate image of the illumination beam.

A preferred embodiment of the beam conjugated approach may include the following features. First, a small beam at the corneal plane is used for the illumination. The beam size should be small enough so that the image of the illumination beam covers very few lenslets in the wavefront sensor plane. Second, a small opaque stop, comparable to the illuminated area at the corneal plane, is placed and bound to an optical flat whose position is adjustable in three axes (x-y-z) in the initial system setup. Along the optical axis, the opaque stop is positioned in the plane conjugate or the lenslet array or next to the lenslet array. In the plane perpendicular to the optical axis, the opaque stop is positioned to block the corneal reflex of the illumination beam and a few fixed lenslets around the optical axis. Alternatively, an opaque stop can be placed and bound to the lenslet array. Third, the small opaque stop will only block wavefront measured at a limited number of lenslets. Wavefront slopes at those missing points can be interpolated or extrapolated according the wavefront slopes next to the missing sampling locations.

Even though our vertex-centered and beam-conjugate methods works fine for both on-vertex illumination and off-vertex illumination, on-vertex illumination is preferred because it will be less sensitive to position errors for the opaque stop. The vertex-centered reflex rejection works better for an illumination beam size larger than 1 mm while the beam-conjugated reflex rejection works better for a small illumination beam size less than 1 mm. Both vertex-centered and beam-conjugated reflex rejections are tolerable to beam position to the cornea vertex because the off-vertex illumination works just fine as the on-vertex illuminations.

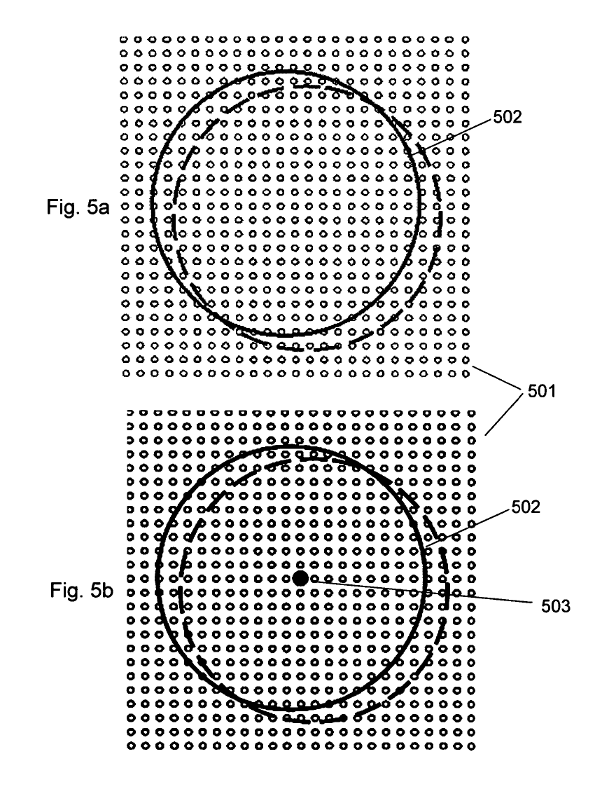

Wavefront sensors using a Hartmann-Shack sensor measure wave aberration by converting phase errors across pupil of an eye to displacements of focus spots between a reference image and an image from a test object. Sensing wavefronts requires two measurements: one reference measurement from a known reference such as a perfect plane wave and one measurement from a tested object. If a large unrestricted lenslet array is used for wavefront measurement, unique registration of each focus spot to its corresponding lenslet is almost impossible as shown in FIG. 5a. The boundary of the human pupil, shown as the circles 502, usually determines the specific region of lenslets 501 for the wavefront measurements. Without a registration mark in the lenslet array, it is not possible to identifying each focus spot to its corresponding lenslet. We propose to introduce a fix, localized feature on the lenslet array to address the registration issue of focus spots and lenslets.

One preferred embodiment for making a registration mark in the lenslet array is to block at least one lenslet in an otherwise unrestricted 2 dimensional lenslet array as shown in FIG. 5b. It can be achieved by placing a small opaque stop on the lenslet array to block only a few lenslets or by placing a small opaque stop on an optical flat that is placed next to or in a plane conjugate to the lenslet array.

Removing systematic wavefront error is possible when all focus spots in the wavefront measurement are correctly registered to the corresponding lenslets. It can be achieved using the following steps. First, a background measurement with a known wavefront such a plane wave coving a large pupil area is taken as the reference. Second, focus spots in the reference image are uniquely registered to the corresponding lenslets according to a localized feature such as the missing lenslets, and the coordinates of each lenslet for the reference wavefront are stored as the reference coordinates. Third, wavefront slopes for the measured eye at each lenslet are derived from the difference between the corresponding focus spots in the reference and in the wavefront measurement of the eye. Wave aberration of the eye with background error removed can be obtained by reconstructing the wavefront from the obtained wavefront slopes.

Another advantage of unique identification of focus spots to their corresponding lenslets is the accurate registration of the detected wavefront map to the natural pupil of the eye. In a typical wavefront measurement, pupil images as well as wavefront sensor images are obtained with two separate cameras. Because coordinates of the lenslet array and the pupil camera can be precisely determined, wavefront map can be accurately registered to natural pupils of eyes if the obtained wavefront is precisely registered to the lenslet array. Ability to register the measured wavefront to the natural pupil of an eye is critical to the success of a wavefront guided vision correction such as laser vision corrections.

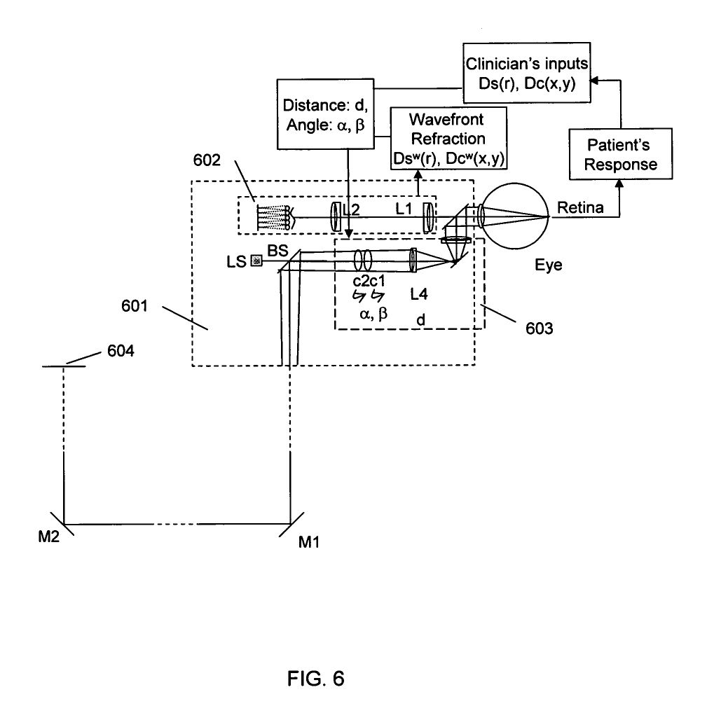

Conventional wavefront sensors usually measure wave aberration of an eye at one accommodation state. Because human eyes do not necessarily accommodate at its far accommodation state during a wavefront measurement, refractive corrections based on the wavefront along can be problematic. FIG. 6 shows an apparatus capable of providing wavefront of an eye at its far accommodation point in accordance to the present invention. The apparatus comprises a wavefront module 602 configure for measuring wave aberration of the eye at one accommodation state, a refraction module 603 configured for determining a manifest sphero-cylindrical refraction of the eye subjectively at the far accommodation state, a wavefront fusion algorithm for determining the wave aberration of the eye at its far accommodation point as described in U.S. patent application Ser. No. 11/432,273, titled “Wavefront Fusion Algorithms for Refractive Vision Correction and Vision Diagnosis,” filed on May 10, 2006 by J. Liang.

The wavefront module 602 provides a conventional objective wavefront measurement. A narrow illumination beam from a light source LS produces a compact light source. The probing light is diffusely reflected by the retina, from which a distorted wavefront is formed at the eye's cornea plane. An optical relay system, consisting of lenses (L1) and (L2), relays the outgoing wavefront from the eye through the beamsplitter to the plane of a lenslet array. A Hartmann-Shack wavefront sensor, consisting of a lenslet array and an image sensor, provides measurement of wave aberration in the eye.

The refraction module 603 provides corrections of defocus and astigmatism in the eye. In a preferred embodiment, two cylindrical lenses have the cylindrical power of about −3D at the eye's cornea. By rotating the two cylindrical lenses to angles of α and β, respectively, the cylindrical lenses can generate astigmatic correction of up to −6D in any direction plus a focus error Dₛ^{A}(r). By changing the distance (d) between two spherical lenses, the refraction module can generate correction for eye's sphero-cylindrical corrections. The settings of the refractive corrections (α, β, d) are first determined based on a wavefront sphero-cylindrical correction in the tested eye determined from the wave aberration from the wavefront sensor, and further controlled by operators based on patient's reading of a distant (>3 meters) acuity target 604. Manifest refraction as well as visual acuity of the eye is measured using an iterative strategy in standard optometric practice.

The wavefront fusion algorithm, described in U.S. patent application Ser. No. 11/432,273, titled “Wavefront Fusion Algorithms for Refractive Vision Correction and Vision Diagnosis,” filed on May 10, 2006 by J. Liang, provides wave aberration of the tested eye at its far accommodation state by combining the wave aberrations measured with the wavefront module and the manifest refraction from the refractive correction module. First, a wavefront spherical error and cylindrical error is determined from the measured wave aberration of the eye. Second, wave aberration at the far accommodation point of an eye is determined by adding an accommodation offset to the measured wave aberration. The accommodation offset is the difference between the manifest spherical power and the wavefront spherical power.

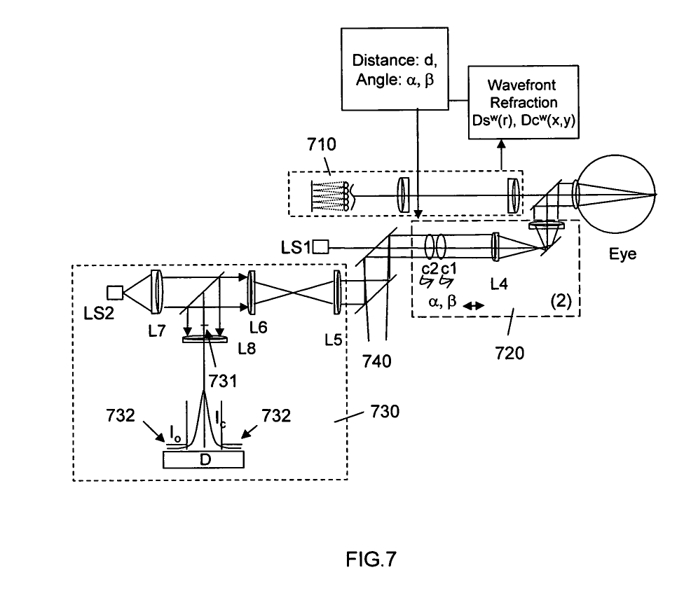

Even though wavefront sensors measures all aberrations in an eye, it still cannot provide a complete description of eye's optical performance because light scattering in the eye is not measured by a conventional wavefront sensor. Light scattering in the eye is caused by scattering centers at microscopic scale and can produce image blur similar to aberration-induced blur. The image blur caused by aberrations distributes in the central portion of the PSF whereas the light scattering spread light blur to a long pedestal in the eye's point-spread function. Westheimer et al described an Index of Light Diffusion (ILD) for the assessment of light scattering in the eye in “Evaluating diffusion of light in the eye by objective means” Investigative Ophthalmology & Visual Science, vol. 35, p2652, 1994. By incorporating an improve measurement of ILD, FIG. 7 shows a wavefront based apparatus capable of measuring not only wave aberration but also light scattering in the eye.

A preferred embodiment of the apparatus comprises a wavefront sensor module 710 configured for measuring wave aberration of the eye, wherein the wave aberrations is represented by a wavefront refraction (the sphero-cylindrical errors) and high-order aberrations in the eye, a refractive correction module 720 configured for correcting the conventional sphero-cylindrical errors based on the wavefront refraction from the wavefront module, a double-pass module 730 configured for measuring light scattering in the eye based on a double-pass measurement of eye's point-spread distribution.

The preferred metrics for measuring light scattering in the eye is the Index of Light Diffusion (ILD) proposed by Westheimer et al. As shown in FIG. 7, a beam from a compact Light Source (LS2) at the focal plan of the lens L7 is focused at the eye's retina. The reflected light from the retina is imaged at the focal plane of the lens L8, and forms a double-pass point-spread function for the eye. Index of Light Diffusion is measured as the ratio of the light energy at an outer region of the double-pass point spread function (Iₒ) to the energy at a central region of the double-pass point-spread function (I_{c}), i.e., ILD=Io/Ic.

A number of improvements are introduced beyond the method proposed in Westheimer et al. First, the ILD measurement is performed after an effective correction for both spherical and astigmatic error in the eye. More particularly, the sphero-cylindrical correction is measured with a wavefront sensor and the sphero-cylindrical correction is achieved by a sphero-cylindrical correction module 720. The effective correction for both the spherical error and the astigmatic error is critical for the ILD measurement because it can ensure that the light energy outside the central region (Io) in the double-pass PSF are indeed due to light scattering only. Second, measurements of ILD are achieved without the influence of the corneal reflection. The method of vertex-centered reflex rejection is incorporated into the ILD measurement using an opaque stop 731. The lens pair (L5 and L6) reproduces the corneal reflection at the opaque stop 731 through a beamsplitter. Third, the ILD measurement is obtained using one light detector (D) with apertures of variable sizes 732. One detector instead of a CCD image sensor is cheaper and can measure the light in the central double-pass PSF (I_{c}) with a smaller aperture while measures the total light in the double-pass PSF (Iₜ) with a larger aperture (or opened completely). ILD of the eye can be derived as (Iₜ−I_{c})/I_{c}. Fourth, the ILD measurement can be further improved by using a modulated light source (LS2) so that the ambient background light can be removed by filtering out the DC components in the electric signal from the detector. Fifth, the ILD can be measured at a series of different focus settings that is achieved by setting different focus through the sphero-cylindrical correction module 720, and the smallest ILD is selected as the final measurement of the light diffusion in the eye. Using the smallest ILD through focus guarantees the best correction of eye's focus error, which can be different from the wavefront sphero-cylindrical correction.

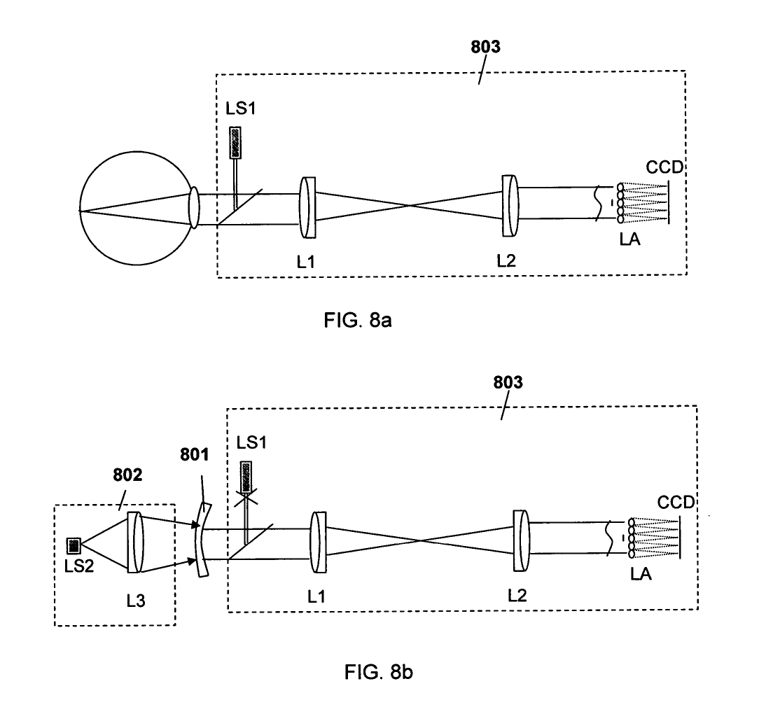

Wavefront sensors measures wave aberrations of eye for refractive correction and vision diagnosis. Building a combined lensometer and a wavefront sensor is highly desired in clinical settings. First, a combined system requires less office space and can be cheaper than two separate systems. Second, measuring lenses with a wavefront sensor allows evaluations of correction lenses beyond the conventional sphero-cylindrical correction. FIG. 8 shows a construction of a lensometer as an addition to a Hartmann-Shack sensor for measuring wave aberration in the eye. The combined system uses one Hartmann-Shack sensor and one optical relay.

When the system is used for measuring aberrations in an eye as shown in FIG. 8a, the wavefront system 803 comprises a light source LS1 configured to produce a compact light source at the retina of an eye if an eye is measured, an optical relay system (L1 and L2) configured to reproduce the measured wavefront to a plane with a wavefront sensor, a wavefront sensor configure to measure the wavefront. The wavefront sensor is a Hartmann-Shack sensor consisting of a lenslet array and an image sensor.

When the system is used for measure a lens as a lensometer as shown in FIG. 8b, the light source in the wavefront system 803 is turned off. Another light source (LS2) produces a wavefront by lens L3 through the lens under test 801. The same optical relay (L1 and L2) and the Hartmann-Shack sensor are used to measure the wavefront from the lens under test. The lensometer contains the following advanced features. First, the preferred illumination for the lensometer is an illumination source outside the wavefront refractor, which creates a wavefront through the tested lens for the wavefront test while the reflections from the lens surfaces do not enter the wavefront refractor. Second, a converging wavefront from 802 is used for the illumination of the tested lens. The converging illumination makes a wavefront refractor, designed to measure eyes with a spherical correction error between −6D (farsighted eyes) to +12D (near sighted eyes), suitable to measure correction lenses with a spherical correction between −12D and +6D. Third, quality of human vision under the tested correction lens can be assessed and specified from the wave aberration of the eye and the wavefront data for the lenses.

A number of embodiments have been described. Nevertheless, it will be understood that various modifications may be made without departing from the spirit and scope of the inventions. For example, advantageous results still could be achieved if steps of the disclosed techniques were performed in a different order and/or if components in the disclosed systems were combined in a different manner and/or replaced or supplemented by other components. Accordingly, other embodiments are within the scope of the following claims.

What is claimed is:

1. A wavefront system for determining wave aberration of an eye, comprising:
an illumination light source configured to produce a compact light source at the retina of the eye;
a small opaque stop configured to block corneal reflection of the illumination light;
a wavefront sensor configured to measure the wavefront originated from the compact light source at the retina.

2. The system of claim 1, wherein the small opaque stop is positioned at a location where the corneal reflection is concentrated to an image point in the wavefront system.

3. The system of claim 2, wherein the small opaque stop is position in a plane that is optically conjugated to the focal plane of cornea surface.

4. The system of claim 1, wherein the small opaque stop is positioned to block an image of the illumination beam at the cornea.

5. The system of claim 4, wherein the small opaque stop is placed in a plane that is optically conjugated to the cornea of the eye.

6. The system of claim 1, wherein the small opaque stop is made of an optical flat and an opaque stop placed on the optical flat.

7. The system of claim 1, wherein the wavefront sensor is a Hartmann-Shack wavefront sensor.

8. The system of claim 7, wherein the small opaque stop is placed on the lenslet array of the Hartmann-Shack wavefront sensor.

9. The system of claim 1, further include determining wave aberration of an eye at its far accommodation point, comprising:
a refractive correction module configured for determining a manifest refraction of the eye subjectively;
a wavefront fusion algorithm for determining the wave aberration of the eye at its far accommodation point by combining the measured wavefront aberration from the wavefront module and the manifest refraction from the refractive correction module.

10. The system of claim 1, further include measuring light scattering in an eye; comprising:
a refractive correction module configured for correcting conventional sphero-cylindrical errors based on the wavefront data from the wavefront sensor;
a double-pass module configured for measuring a double-pass point-spread distribution of the eye; and
specifying light scattering in the eye based on the double-pass measurement.

11. The system of claim 7, is further configured as a lensometer for measuring a lens for refractive correction, comprising:
a separate light source to produces a wavefront through the tested lens while the illumination light source for measuring the eye is turned off;
a mechanical subsystem for holding the lens;
measuring wavefront of the refractive lens using the same Hartmann-Shack sensor for the eye;
specifying the lens based on the measured wavefront data.

12. A method of wavefront sensing of human eye with a Hartmann-Shack sensor, the method comprising the steps of:
producing a compact light source at retina of the eye;
receiving the light reflected from the retina with a Hartmann-Shack sensor, wherein the Hartmann-Shack sensor includes a fixed, localized feature for unique identification of each focus spot in the wavefront image to its corresponding lenslet;
determining coordinates of focus spots in the wavefront image;
calculating wavefront slopes from the displacements of focus spots;
constructing wave aberration of the eye from the calculated wavefront slopes.

13. The method of claim 12, wherein the fixed, localized feature in the Hartmann Shack sensor is realized by blocking at least one lenslet in the two-dimensional lenslet array for wavefront sensing.

14. The method of claim 12, wherein determining coordinates of focus spots in the wavefront image comprises the steps of:
identifying focus spots in the wavefront image;
determining the coordinates of each focus spot;
registering each focus spot to its corresponding lenslet in the lenslet array based on the fixed, localized feature in the Hartmann-Shack sensor.

15. The method of claim 12, wherein calculating wavefront slopes from displacements of focus spots comprises the steps of:
obtaining coordinates of involved lenslets in a reference image, wherein the reference image is obtained by measuring a perfect known wavefront such as a plane wave;
calculating the displacements of each focus spot in x- and y-directions from the coordinates in the reference image and those in wavefront measurement of an eye;
deriving wavefront slopes in x- and y-direction for each sampling points as a ratio of the calculated displacements to the focal length of the lenslet array.

16. The method of claim 12, further includes registering wavefront distribution across the pupil of the eye based the fixed, localized feature in the Hartmann-Shack sensor and an image of the eye's pupil.

17. The method of claim 16, further includes correcting an optical error of an eye, comprising:
a processor for generating an ablation pattern of laser energy for ablation of a corneal tissue of the eye so as to correct the measured optical error, the ablation pattern based at least in part on the measured wave aberration of the eye; and
a laser system for directing laser energy onto the corneal tissue of the eye to achieve the generated ablation pattern.

18. An apparatus for determining a wave aberration of an eye at its far accommodation point, comprising:
a wavefront module configure for measuring wave aberration of the eye;
a refraction correction module configured for determining a manifest refraction of the eye subjectively, wherein the manifest refraction comprises of at least a manifest spherical power;
a wavefront fusion algorithm for deriving the wave aberration of the eye at its far accommodation point by combining the measured wavefront aberration from the wavefront module and the manifest refraction from the refraction correction module.

19. The apparatus of claim 18, wherein the wavefront module comprises:
producing a compact light source at the retina of the eye;
receiving the light reflected from the retina with a detector; and
detecting a wave aberration of the eye with the detector like a Hartmann-Shack sensor.

20. The apparatus of claim 18, wherein the refraction correction module comprises:
presenting an acuity chart at a distance of about 3 meters to 6 meters away to the tested eye;
measuring visual acuity of the eye subjectively with a correction for the conventional sphero-cylindrical error by varying the distance between 2 spherical lenses and the orientations of two cylindrical lenses;
determining a manifest refraction for the eye at the far point of the eye based on subjective feedbacks of the tested patient using a recursive process.

21. The apparatus of claim 20, wherein the true distance of about 3 meters to 6 meters is achieved by placing at least one mirror between the tested eye and the acuity chart for reduced room space.

22. The apparatus of claim 18, wherein the wavefront fusion algorithm for deriving a wave aberration of the eye at its far accommodation point comprises the steps of:
determining a wavefront refraction for the spherical and cylindrical powers from the wave aberration of the eye;
determining a wave aberration of the eye at its far accommodation point by adding an accommodation offset to the measured wave aberration, wherein the accommodation offset is the difference between the manifest spherical power and the wavefront spherical power.

23. An apparatus for measuring wave aberration and light diffusion in an eye, the apparatus comprising:
a wavefront module configured for measuring wave aberration of the eye, wherein the wave aberrations is represented by a wavefront refraction (a sphero-cylindrical correction) and high-order aberrations in the eye;
a refractive correction module configured for the conventional sphero-cylindrical correction based on the wavefront data from the wavefront module;
a double-pass module configured for measuring double-pass point-spread distribution of the eye;
a metrics for qualifying the light diffusion in the eye based on the data from the double-pass module.

24. The apparatus claim of 23, wherein the wavefront module is a Hartmann-Shack sensor for the eye comprises:
a fixation target configured for stabilizing accommodation of the eye;
an illumination light source configured to produce a compact light source at the retina of the eye; and
a Hartmann-Shack sensor configured to measure the wavefront originated from the compact light source at the retina of the eye.

25. The apparatus claim of 23, wherein a refractive correction module is achieved by varying the distance between 2 spherical lenses and the orientations of two cylindrical lenses.

26. The apparatus claim of 23, wherein the double-pass module comprises:
a light source configured to produce a compact light illumination at the retina like a point-spread distribution;
an imaging module configured to produce an optical image of the light distribution at the retina; and
a light detector for measuring the double-pass retinal image.

27. The apparatus claim of 23, further comprises an small opaque stop to block unwanted reflections from the cornea of the eye.

28. The apparatus claim of 26, wherein the detector comprises a photocell that converts the photons in the double-pass image into an electric signal and an aperture that controls the effective size of the double-pass image exposed to the photocell.

29. The apparatus claim of 26, wherein the light source is time modulated and the signal of the detector is filtered to removal the contribution of ambient light.

30. The apparatus claim of 23, wherein the metrics for qualifying the light diffusion in the eye is a ratio of two integrated intensities of the double-pass point-spread distribution.

31. The apparatus claim of 30, wherein the two integrated intensities of the double-pass point-spread distribution are the total energy in an inner circular region and the total energy in an outer annular region of the double-pass point-spread distribution.

32. The apparatus claim of 30, wherein the two integrated intensities of the double-pass point-spread distribution are the total energy of the double-pass point-spread distribution and the energy in the central portion of the double-pass point-spread distribution.

33. An apparatus for measuring wave aberration of an eye and for measuring lenses as a lensometer using one Hartmann-Shack sensor, the apparatus comprising:
a light source configured to produce a compact light source at the retina when an eye is measured;
a second light source configured to produce a wavefront through a lens when the lens is measured;
an optical relay for transferring the measured wavefronts to a plane with a wavefront sensor;
a Hartmann-Shack sensor for measuring either a wavefront from an eye under test or a wavefront through a lens under test.

34. The apparatus of claim 33, further include specifying performance of the eye under test based on the wavefront measured from the eye.

35. The apparatus of claim 33, further include specifying parameter of the lens under test based on the measured wavefront trough the lens.

35. The apparatus of claim 33, further include specifying quality of a lens based on the measured wavefront from the eye under test and the wavefront trough the lens under test.
